(19) United States
(12) Patent Application Publication
(10) Pub. No.: US 2003/0091650 A1
(43) Pub. Date: May 15, 2003

(54) Ratite oil as a topical adjuvant and transdermal carrier

(76) Inventor: Linda Wells, Northridge, CA (US)

Correspondence Address: Linda Wells, 9013 Amigo Ave., Northridge, CA, 91324, US

(21) Appl. No.: 10/010,771

(22) Filed: Nov. 13, 2001

(51) Int. Cl. A61K 35/12; A61K 47/00; A61K 35/78

(52) U.S. Cl. 424/522; 514/786; 424/730

(57) ABSTRACT

My invention is the use of ratite oil as a topical adjuvant and as a transdermal delivery vehicle to carry agents, especially medicines, into the bodies of mammals, including humans. This patent application specifically excludes progesterone as one of the claimed agents (U.S. Pat. No. 6,303,132), and the topical use of plain emu oil (patent application 20010033838).

### CROSS REFERENCE TO RELATED APPLICATIONS

| | | | |
|---|---|---|---|
| 6303132 | 5662921 | 5431924 | 4150128 |
| 5472713 | 5677338 | 5688746 | 5698227 |
| 5725858 | 5744128 | 5747659 | 5786179 |
| 5801196 | 5849334 | 6056972 | 5626882 |
| 5929113 | | | |

### FEDERALLY SPONSORED RESEARCH/DEVELOPMENT

No federal funds were used by the inventor in the creation of this invention.

### BACKGROUND OF THE INVENTION

It all started with my father, an intelligent and curious engineer who loves to tour factories, ranches, nature preserves; family outings are invariably educational. During a week long visit, I took him to Mourning Cloak Botanical Gardens for a tour. To my horror, I heard one of the staff recommending DMSO as a carrier for some of their alternative medicinal plant preparations. Although efficacious, DMSO has toxic side effects, and is not recommended for human use. The next day we were touring ostrich, emu, and rhea ranches, when I overheard a ranch hand complain about how ostrich oil went right through his gloves, which my quick glance revealed were heavy leather. As a research scientist, I am very sensitive to proper glove usage for different toxins for adequate skin protection, as my life depends upon a successful choice of material. It occurred to me immediately that if this oil could easily penetrate old, thick leather, it should effectively penetrate human skin, and could act as a transdermal carrier. Further, ostrich oil is nontoxic, unlike DMSO, so I was hopeful that it could be utilized as a safe and efficacious carrier for medicinal compounds, both alternative and traditional.

To test this idea, I decided to make a cream with ostrich fat and herbal anti-inflammatories to treat the carpal tunnel in my wrists. I have suffered from pain in my wrists from some years, and was diagnosed by a Kaiser physician with carpal tunnel. First I purchased some ostrich fat from a processing plant and rendered the oil from the fat in my crockpot. I mixed it with other ingredients to make a nice cream, then added comfrey root tincture, thyme, and turmeric as active ingredients, which are herbal medicines reputed to reduce inflammation. I applied the cream to the inside of my wrists three times daily. During the next few weeks, my pain slowly but steadily decreased, until after six weeks I was free of pain. The carpal tunnel syndrome does recur with overuse of my hands or extended vibration, but the cream works quite well for reduction of the pain and symptoms. It is certainly preferably to the only other option - surgery. For commercial applications, I would definitely omit the turmeric, as it stains everything it contacts a brilliant yellow. I also gave some of the cream to an woman whose elderly husband was in constant pain from arthritis. I explained how to test for an allergic reaction before use, then told her to apply the cream three times/day on the painful joints. She reported that he did experience some relief from pain, although there was not a complete cure.

I next made a lotion for my father, who was complaining about dry, itchy skin. I used the ostrich fat as a topical adjuvant to increase the moisturizing and anti-microbial activity of the herbal extractions. I mixed the ostrich fat with other moisturizing ingredients, freshly extracted aloe gel to further sooth the skin, and homemade, fresh lavender tincture for a pleasant odor and antimicrobial activity. My father used the lotion and reported the itching cleared up immediately, and the skin was moisturized for at least 24 hours before reapplication was required. At this point, I decided the invention was efficacious and beneficial, and I would apply for a patent.

After doing some research in the patent data base, I found that past patents have concentrated on emu oil. This embodiment claims ratite oil, excluding all claims made by previous inventors of emu oil. I have several reasons for the invention of ratite oil. Dr. Ernesto Hernandez, a biochemist at the Food Protein Research and Development Center of Texas A & M University, has found that, “The oils obtained from all three species of ratites, namely emu, ostrich and rhea appear to be similar in the basic composition. The oils are triglycerides composed mostly of oleic, palmitic, steric, and linoleic fatty acids.” The fatty acids are very similar to each other biochemically, as diagrammed by Stryer in his biochemistry text. Dr. Hernandez found that the proportions of the oils are also very similar between the ratites. The ostrich oil rendered by my method sank into the skin extremely quickly and showed the same noncomedegenic properties of the emu oil, not surprising considering their biochemical similarities. It is thought that the oils penetrate the skin so well because they are not phosphorylated, and thus can permeate the epidermis quickly and efficiently. However, while I was visiting the ostrich and emu ranches, I discovered that the emu ranchers were converting to ostrich ranching. The meat is more popular among consumers, hence ostrich ranching is more profitable in the United States. In addition, emus have a defense mechanism whereby they flip in midair and eviscerate predators/ranch hands on the way down, which does not endear them to ranch personnel. Ostriches and rhea are safer and more profitable to rear, thereby creating a much larger market of fat for medicinal purposes. Ostriches are larger than emus and certified processing plants for ostriches are already available in the States, making them a superior source of oil.

Ratite oils have wonderful properties in and of themselves. Some of these properties have already been claimed for emu oil. This invention differs from patent application 20010033838 in that the remaining ratite oils are claimed for topical delivery and topical adjuvant purposes. In addition, the previous patent application claims only topical carrier functions for emu oil, while my application claims transdermal carrier properties for emu oil. My application also differs from previous claims for emu oil for cosmetic and anti-aging use in that the ratite oil is used as an adjuvant for other agents. Lidocaine (U.S. Pat. No. 5,849,334) and anesthetics (U.S. Pat. No. 5,698,227) are also specifically excluded for the topical adjuvant claim, as they are protected by previous patents.

Transdermal delivery systems have several advantages over oral medications. Stomach acid inactivates many otherwise useful drugs; transdermal delivery bypasses this problem. Gut absorption of medicinal compounds is often quite inefficient, and topical application provides another avenue that is more efficacious for some active ingredients. Additionally, creams and lotions can be applied directly to the problem areas, maximizing medicinal concentrations where needed by the patient. Some medicines have a foul taste; some patients have trouble swallowing and/or retaining pills; some patients dislike shots and needles. Topical delivery provides another delivery option for the pharmaceutical industry, to maximize patient health and comfort. My application embodies ratite oil as a transdermal delivery mechanism for herbal and traditional medicines, compounds, and agents, specifically excluding the use of emu oil as a transdermal delivery mechanism for progesterone, as protected by U.S. Pat. No. 6,303,132.

### BRIEF SUMMARY OF THE INVENTION

The intent of this patent application is to claim all uses of ratite oil as a topical adjuvant and as a transdermal carrier as broadly as possible, to protect the profitability of commercial applications in the future. It is also the intent of the inventor to exclude claims protected by previous patents.

The purpose of this invention is to patent and protect, for commercial applications, a safer and more effective medical transdermal delivery system and a novel topical adjuvant, namely ratite oil, excluding plain emu oil as a topical adjuvant and progesterone as an agent, as protected by previous patents. The optimal delivery method for pharmaceuticals differs from drug to drug, patient to patient, and application to application. This invention adds another safe and effective drug delivery system to the pharmaceutical arsenal.

### DETAILED DESCRIPTION OF THE INVENTION

The foregoing description of the specific embodiments of the present invention illuminates a superior topical adjuvant and transdermal delivery system, i.e., ratite oil. Although particular formulations will be cited as examples, these are for the purpose of illustration only, and they are not intended to be limiting with respect to the scope of my claims. I predict that many substitutions, modifications, derivations, fractionations, and alterations will be made to the invention without departing from the spirit and scope of the invention as defined by the claims. In particular, the final form of the invention (gel, cream, lotion, emulsion, oil, ointment, suspension, aerosol, spray, powder, aerosol powder, or semi solid formulation, etc.) and the delivery system (pump dispenser, jar, spray bottle, pouch, tube, roll-on, squeeze bottles, flexible articles intended to be worn on the skin, impregnating said composition into a fibrous or non-fibrous matrix, dermal patch, adhesive tape, etc.) are believed to be a matter of routine for a person of ordinary skill with knowledge of the embodiments described herein. The pharmaceutical methodology of making topical therapeutics is well known, hence it will not be described in detail in this application. Only the aspects specific to the invention will be elucidated in this application.

In general, in one aspect, the invention relates to a medicinal composition comprising a concentration of ratite oil and a concentration of an agent or medicinal compound.

In general, in another aspect, the invention refers to a method of topically administering a medicinal composition. The method comprises applying to the skin surface a composition containing a concentration of ratite oil and a concentration of an agent or medicinal compound.

The embodiments herein are illustrative only, and other embodiments may be derived by one having ordinary skill without departing from the scope of the invention. For example, the formulations are anticipated to include, but are not limited to, gel, cream, lotion, emulsion, oil, ointment, suspension, aerosol, spray, powder, aerosol powder, or semi solid formulation, etc., as obvious pharmaceutical applications for a person of ordinary skill with knowledge of the embodiments described herein. The composition may be packaged in any manner of suitable dispenser types that are obvious in origin, including but not limited to pump dispenser, jar, spray bottle, pouch, tube, roll-on, squeeze bottles, flexible articles intended to be worn on the skin, impregnating said composition into a fibrous or non-fibrous matrix, dermal patch, adhesive tape, etc. The therapeutic dosage may be applied as often and in any dosage as may be necessary for an effective treatment of symptoms or illness. The optimal percentages of ratite oil and active ingredient will differ for each active ingredient. The details of the embodiments of this invention are set forth in the description below. In this specification and my claims, the singular forms include plural referents unless the context clearly dictates otherwise. All examples are for illustration purposes only. Therefore, the scope of the invention should be limited only by the claims.

Quick penetration is critical for efficacy of some medicines and agents, as active ingredients decompose on the skin surface, and in addition are subject to removal. It is very desirable, therefore, to have a carrier that is absorbed very quickly. Ratite oils absorb extremely quickly, and hence are a superior transdermal delivery mechanism. Not only are they non-toxic and noncomedegenic, but they are beneficial in and of themselves, exhibiting antibacterial, antioxidant, and moisturizing properties. Ratite oil is relatively inexpensive and readily available. Hence, I seek to patent and promote the use of ratite oil as a topical adjuvant and transdermal delivery system.

The trial formulations that I've composed to date, intended to serve as examples and not to limit the invention, are:

Anti-inflammatory cream for carpal tunnel and arthritis

¼ cup ostrich oil

2 Tbls. beeswax

1 Tbls. each almond oil and vitamin E oil

Melt the above ingredients together and keep warm.

Mix and warm the following ingredients:

2 Tbls. comfrey tincture

1 Tbls. each ground thyme, and turmeric

1 teas. benzoin

Heat both the lipid-based mixture and the aqueous mixture gently to approximately the same temperature. Pour at once into a commercial mixer and beat for 30 minutes. Apply to painful joints or wrists three times daily.

This formulation slowly eliminated the pain from my carpal tunnel syndrome, and somewhat eased the pain from arthritis in an elderly man.

In another embodiment of the invention, ostrich oil was used as a topical adjuvant.

Topical Lavender Lotion

¼ cup rendered ostrich oil

¼ cup beeswax

2 Tbls. each lanolin, almond oil, and Vaseline

1 Tbls. vitamin E oil

Melt the ingredients above together and keep warm.

Mix the following ingredients and gently heat until approximately the same temperature as the lipid mixture:

¼ cup aloe gel

¼ c fresh lavender tincture

1 teas. Benzoin

2 drops yellow food coloring

Pour both mixtures at once into a commercial mixer and beat for thirty minutes. Apply daily to dry, itchy skin. Keep refrigerated.

My father used this lotion and noted that his skin was not dry or itchy for 24 hours. This lotion was then used by two other people, who reported that their skin was not dry for 24 hours. Lavender and aloe sooth the skin and have anti-microbial properties.

Based on these successes, I decided to patent and promote the use of ratite oil as a topical adjuvant and transdermal delivery system.

What is claimed is:

1. The use of ratite (ostrich, emu, rhea, cassowary) oil as a topical adjuvant for agents on mammalian skin, in any formulation including but not limited to gel, cream, lotion, emulsion, oil, ointment, suspension, aerosol, spray, powder, aerosol powder, or semi solid formulation, etc., in a container or dispenser, including not limited to pump dispenser, jar, spray bottle, pouch, tube, roll-on, squeeze bottles, flexible articles intended to be worn on the skin, impregnating said composition into a fibrous or non-fibrous matrix, dermal patch, adhesive tape, etc. This claim specifically excludes progesterone (U.S. Pat. No. 6,303,132).

2. The composition of claim 1, wherein any fraction, derivation, alteration, or modification of ratite oil is used in the preparation in any concentration.

3. The composition of claim 1, wherein the agent includes, but is not limited to, alternative medicines, such as St. John's Wort or comfrey root, and traditional medicines, such as ibuprofen, whether in purified, partially purified, or unpurified form, in any concentration, specifically excluding Lidocaine (U.S. Pat. No. 5,849,334) and anesthetics (U.S. Pat. No. 5,698,227).

4. The use of ratite oil as a transdermal delivery system for agents on mammalian skin, in any formulation including but not limited to gel, cream, lotion, emulsion, oil, ointment, suspension, aerosol, spray, powder, aerosol powder, or semi solid formulation, etc., in any container or dispenser, including but not limited to pump dispenser, jar, spray bottle, pouch, tube, roll-on, squeeze bottles, flexible articles intended to be worn on the skin, impregnating said composition into a fibrous or non-fibrous matrix, dermal patch, adhesive tape, etc. This claim specifically excludes Lidocaine (U.S. Pat. No. 5,849,334) and anesthetics (U.S. Pat. No. 5,698,227).

5. The composition of claim 4, wherein any fraction, derivation, alteration, or modification of ratite oil is used in the preparation in any concentration.

6. The composition of claim 4, wherein the agent includes, but is not limited to, alternative medicines, such as St. John's Wort or comfrey root, and traditional medicines, such as ibuprofen, whether in purified, partially purified, or unpurified form, in any concentration, specifically excluding progesterone (U.S. Pat. No. 6,303,132).

7. A method of topically administering agents and ratite oil, comprising application of a composition of ratite oil and an agent, to the skin surface of mammals, in any type of formulation.

8. The method in claim 7, wherein any fraction, alteration, modification, or derivation of ratite oil is utilized in the product in any concentration.

9. The method in claim 7, wherein any agent is delivered transdermally using ratite oil, in any concentration.

10. The method in claim 7, wherein the composition may be any type of formulation, including but not limited to gel, cream, lotion, emulsion, oil, ointment, suspension, aerosol, spray, powder, aerosol powder, or semi solid formulation, etc.

11. The method in claim 7, wherein the composition can be packaged for sale in a container or dispenser, including but not limited to, pump dispenser, jar, spray bottle, pouch, tube, roll-on, squeeze bottles, flexible articles intended to be worn on the skin, impregnating said composition into a fibrous or non-fibrous matrix, dermal patch, adhesive tape, etc.
